(19) United States
(12) Patent Application Publication
(10) Pub. No.: US 2024/0029820 A1
(43) Pub. Date: Jan. 25, 2024

(54) COMPUTING AFFINITY FOR PROTEIN-PROTEIN INTERACTION

(71) Applicants: Nant Holdings IP, LLC, Culver City, CA (US); ImmunityBio, Inc., Culver City, CA (US)

(72) Inventors: Bing Song, La Canada, CA (US); Shiho Tanaka, Redondo Beach, CA (US); Clifford Anders Olson, Long Beach, CA (US); Phillip Yang, Los Angeles, CA (US); Patrick Soon-Shiong, Los Angeles, CA (US)

(73) Assignees: Nant Holdings IP, LLC, Culver City, CA (US); ImmunityBio, Inc., Culver City, CA (US)

(21) Appl. No.: 18/225,098

(22) Filed: Jul. 21, 2023

Related U.S. Application Data
(60) Provisional application No. 63/391,704, filed on Jul. 22, 2022.

(51) Int. Cl. G16B 15/30; G16B 15/20

(52) U.S. Cl. CPC G16B 15/30; G16B 15/20

(57) ABSTRACT

Techniques are provided for computing affinity for protein-protein interaction. 3D structure models of the first and second protein parts are generated using a trained first deep learning model. A 3D structure model of a protein-protein complex comprising the first and the second protein parts is generated using a trained second deep learning model. A low energy score state is determined for the 3D structure models of each of the first and second protein parts, and the protein-protein complex. A relax algorithm applied to amino acid side chain and backbone 3D structure models determines a low energy score state for the 3D structure models. Based on the low energy score states, an energy score is generated for the 3D structure models, and a score difference is determined between the energy scores, where the score difference defines a binding affinity score.

### CROSS-REFERENCE TO RELATED APPLICATIONS

This application claims priority to U.S. Provisional Patent Application Ser. No. 63/391,704, filed Jul. 22, 2022, titled “COMPUTING AFFINITY FOR PROTEIN-PROTEIN INTERACTION”. The contents of which is hereby incorporated by reference in their entirety for all purposes.

### TECHNICAL FIELD

This disclosure relates generally to protein-protein interaction, and more specifically to computer model-based predictions used for computing protein-protein interaction affinity.

### BACKGROUND

Proteins consist of chains of amino acids which spontaneously fold, in a process called protein folding, to form the 3D structure of the protein (the 3D structure complex). The 3D structure of a protein, also referred to as its tertiary structure, is made by further folding of secondary proteins, i.e., side chains of amino acids. Interactions between the side chains of amino acids lead to the formation of the tertiary structure, and bonds form between them as the protein folds. The unique amino acid sequence of a protein is reflected in its unique folded structure.

The 3D structure complex is crucial to the biological function of the protein. For example, mutations that alter an amino-acid sequence can affect the function of a protein. Therefore, knowledge of protein's 3D structure is crucial for understanding how the protein works. For example, the 3D structure information can be used to control or modify a protein's function, predict which molecules will bind to a protein, understand various biological interactions, assist in drug discovery, design custom proteins, or other scientifically useful endeavors. Thus, if it is possible to predict protein structure from the amino-acid sequence alone, it would greatly help to advance scientific research.

However, the computation of protein-protein interaction (i.e., binding) affinity is far from mature because understanding how an amino-acid sequence can determine the 3-D structure is highly challenging. The “protein folding problem” involves understanding the thermodynamics of the interatomic forces that determine the folded stable structure, the mechanism and pathway through which a protein can reach its final folded state with extreme rapidity, and how the native structure of a protein can be predicted from its amino-acid sequence.

The classical method of computing protein-protein binding affinity generally starts with an original 3D structure complex (e.g., obtained from the Protein Data Bank archive) to compute a new protein-protein complex structure through the perturbations of the original protein structures in the conformational space (i.e., the space encompassing all possible positions of the protein-protein complex). Next, the energy present in the protein-protein interactions is minimized, such as by a protein-protein docking modelling technique which can predict the structure of a protein-protein complex, given the structures of the individual proteins. However, if the complex structure is flexible, such as in the case of an antigen-antibody interaction, the classical method does not achieve the desired accuracy for both 3D structures and binding affinity.

The classical 3D structure-based method does not need training data and can be characterized as un-supervised learning because the binding affinity can be computed without a priori knowledge of the 3D structure (supervised learning). However, in order to work at all, the classical method may require several thousand training samples that may not be available. As a result of these data gaps, classical methods used for computing an affinity for protein-protein interaction are largely unreliable. Oftentimes, computed binding affinity values can be very different between different algorithms and even when different sets of parameters are used for the same algorithm.

Recently, deep learning prediction models have been developed to predict protein structures from a protein's amino-acid sequence alone. Deep learning is a class of machine learning algorithm that uses multiple layers to progressively extract higher-level features from the raw input data. For example, the Baker laboratory at the University of Washington (https://www.bakerlab.org/) runs a physics and statistics-based platform called the Rosetta software suite, which includes algorithms for computational modeling and analysis of protein structures. The Rosetta platform can compute both protein 3D structure and binding affinity. Alphabet, Inc.'s DeepMind has developed the AlphaFold platform, also for predicting a protein's 3D structure from its amino acid sequence. Further, Nantworks' ImmunityBio, Inc. has used its molecular dynamic simulator platform to do similar work. Molecular dynamics simulations allow protein motion to be studied by following their conformational changes through time. Proteins are typically simulated using an atomic-level representation, where all or most atoms are explicitly present. These platforms, e.g., Rosetta, AlphaFold1 and AlphaFold2, UC San Francisco's ESMFold Evolutionary Scale Modeling, etc., have been trained using one or more public repositories of protein sequences and structures that have been assembled over the years. They generally use an “attention network”, a deep learning technique that is meant to mimic cognition attention by enhancing some parts of the input data while diminishing other parts, to first focus on identifying parts of a larger problem, then assemble the parts (e.g., using correlation techniques) to obtain an overall solution. Similar deep learning prediction models, such as DeepAB and ABLooper, have been trained and developed specifically for antibody structure prediction.

### SUMMARY

Systems, methods, and articles of manufacture for computing affinity for protein-protein interaction are described herein. In various embodiments, deep learning models are combined with classical free energy minimization techniques in a novel pipeline to compute protein-protein interaction. For example, a first deep learning prediction model, such as AlphaFold2, DeepAB, ABLooper, etc., can be used to compute 3D structure for a protein part (e.g., an antigen or antibody). The model may use an ensemble of checkpoints or initial random seeds to find the final scores for predictions. A multimer model, e.g., AlphaFold2, can be used to compute 3D structure for an antigen-antibody (Ag+Ab) complex. For example, if the binding site is known, the complex that includes the binding site may be a template input. A relax algorithm, e.g., Rosetta Relax or similar, may be used (Ag, Ab, Ag+Ab) for both side chain and back bone fine tuning of the predicted 3D structure to find a low energy score state and compute an energy score for the 3D structure. The score difference can then be computed between the antigen-antibody complex (Ag+Ab) and the sum of the protein parts (Ag, Ab). This score difference is defined as a binding affinity or binding energy score.

In an embodiment, amino acid sequence data (e.g., FASTA format sequence data from an amino acid sequence database) is obtained corresponding to a first protein part and a second protein part, each comprising flexible complementary-determining region (CDR) loop structures, e.g., an antigen (Ag) and an antibody (Ab). The amino acid sequence data corresponding to the first protein part and the second protein part, respectively, is feed into a trained first deep learning model, where the trained first deep learning model is trained to predict a 3D structure model based on a first input of amino acid sequence data corresponding to a protein part, and 3D structure models of the first protein part and the second protein part predicted by the trained first deep learning model are obtained. The first deep learning model may comprise at least one of the following: AlphaFold2; AlphaFold-Multimer; DeepAB; or ABLooper. For example, the first deep learning model may be trained to use at least one of ensemble checkpoints or initial random seeds for predict the 3D structure models of the first protein part and the second protein part. The amino acid sequence data corresponding to the first protein part and the second protein part is feed into a trained second deep learning model, where the trained second deep learning model is trained to predict a 3D structure model of a protein-protein complex based on a second input of amino acid sequence data corresponding to protein-protein complex parts, and a 3D structure model of the protein-protein complex comprising the first protein part and the second protein part predicted by the trained second deep learning model is obtained. The second deep learning model may comprise at least one of the following: AlphaFold2; AlphaFold-Multimer; DeepAB; or ABLooper, and, therefore, may be the same as the first deep learning model. A low energy score state is determined for the 3D structure models of each of the first protein part, the second protein part, and the protein-protein complex. A relax algorithm, applied to amino acid side chain and backbone 3D structure models of each of the first protein part, the second protein part, and the protein-protein complex, may be used to determine the low energy score state for the 3D structure models of each of the first protein part, the second protein part, and the protein-protein complex. For example, the relax algorithm may comprise at least one of the following: Rosetta Relax or Amber Relax. Based on the low energy score states, an energy score is generated for the 3D structure models of each of the first protein part, the second protein part, and the protein-protein complex; and a score difference is determined between the energy score for the 3D structure model of the protein-protein complex and a sum of the energy scores for the 3D structure models of the first protein part and the second protein part, where the score difference defines a binding affinity score.

In some embodiments, at least one interaction of residue pairs in interfaces between the first and second protein sequences may be selected based on the binding affinity score, and substitution of at least one amino acid of the first or second protein sequences may be facilitated to control a binding affinity for the at least one interaction of residue pairs. The selection of the at least one interaction of residue pairs may be based on at least one of: the at least one interaction comprising a conserved helix structure, a repulsive energy between the potential residue pairs, or a distance between the potential residue pairs.

In some embodiments, substituting the at least one amino acid may comprise substituting an amino acid having a relatively low binding energy with respect to a binding energy mean for a corresponding protein sequence to increase the binding affinity for the at least one interaction of residue pairs. In some embodiments, substituting the at least one amino acid may comprise substituting an amino acid having a relatively high binding energy with respect to a binding energy mean for a corresponding protein sequence to decrease the binding affinity for the at least one interaction of residue pairs.

Various objects, features, aspects and advantages of the inventive subject matter will become more apparent from the following specification, along with the accompanying drawings in which like numerals represent like components.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a visual representation of protein complementary-determining region (CDR) loops in accordance with an embodiment.

FIG. 2A illustrates a block diagram of a data pipeline for computing protein-protein interaction affinity in accordance with an embodiment.

FIG. 2B illustrates an example deep learning model for computing protein-protein interaction affinity in accordance with an embodiment.

FIG. 3 illustrates a block diagram of a system for computing protein-protein interaction affinity in accordance with an embodiment.

FIG. 4 illustrates a flow diagram of example operations for computing protein-protein interaction affinity in accordance with an embodiment.

FIG. 5 illustrates a flow diagram of example operations for computing protein-protein interaction affinity in accordance with an embodiment.

FIG. 6 illustrates a visual 2D representation of a protein complex in accordance with an embodiment.

FIG. 7 illustrates a visual 2D representation of an N803-IL15Rβγ protein complex in accordance with an embodiment.

FIG. 8 illustrates a visual 3D representation of an N803-IL15Rβγ protein complex in accordance with an embodiment.

FIG. 9 illustrates a graphical representation related to increasing binding affinity between IL15/IL15Ra and IL15Rb/IL15Rg in accordance with an embodiment.

FIG. 10 illustrates a graphical representation related to increasing binding affinity between IL15/IL15Ra and IL15Rb/IL15Rg in accordance with an embodiment.

FIG. 11 illustrates a graphical representation related to increasing binding affinity between IL15/IL15Ra and IL15Rb/IL15Rg in accordance with an embodiment.

FIG. 12 illustrates a flow diagram of example operations for increasing protein-protein binding affinity in accordance with an embodiment.

FIG. 13 illustrates a graphical representation related to decreasing binding affinity between IL15/IL15Ra and IL15Rb/IL15Rg in accordance with an embodiment.

FIG. 14 illustrates a graphical representation related to decreasing binding affinity between IL15/IL15Ra and IL15Rb/IL15Rg in accordance with an embodiment.

FIG. 15 illustrates a flow diagram of example operations for decreasing protein-protein binding affinity in accordance with an embodiment.

FIG. 16 illustrates a block diagram of a distributed computer system that can be used for implementing one or more aspects of the various embodiments.

While the invention is described with reference to the above drawings, the drawings are intended to be illustrative, and other embodiments are consistent with the spirit, and within the scope, of the invention.

### DETAILED DESCRIPTION

The various embodiments will now be described more fully hereinafter with reference to the accompanying drawings, which form a part hereof, and which show, by way of illustration, specific examples of practicing the embodiments. This specification may, however, be embodied in many different forms and should not be construed as being limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

The ability to compute the binding affinity of protein-protein complexes is very important for advancing modern drug discovery techniques. Proteins are typically simulated using an atomic-level representation, where all or most atoms are explicitly present. Proteins consist of chains of amino acids which spontaneously fold, in a process known as protein folding, to form the three-dimensional (3D) structures of the protein. These 3D structures are crucial to the biological function of the protein. However, understanding how the amino acid sequences can determine the 3D structure is highly challenging, and is thus commonly referred to as the “protein folding problem”.

Molecular dynamics simulations allow protein motion to be studied by following their conformational changes through time. Unfortunately, the computation of protein-protein binding affinity is far from mature. Oftentimes, the binding affinity value can vary with different algorithms and even with different sets of parameters of the same algorithm.

The classical method of computing protein-protein binding affinity generally starts with an original 3D structure complex (e.g., obtained from the Protein Data Bank archive) to compute a new protein-protein complex structure through the perturbations of the original protein structures in the conformational space (i.e., the space encompassing all possible positions of the protein-protein complex). Next, the energy present in the protein-protein interactions is minimized, such as by a protein-protein docking modelling technique which can predict the structure of a protein-protein complex, given the structures of the individual proteins. However, if the complex structure is flexible, such as in the case of an antigen-antibody interaction, the classical method oftentimes does not achieve the desired accuracy for both 3D structures and binding affinity.

Recent breakthroughs in deep learning technology have addressed the protein folding problem and enabled 3D models of protein structures to be predicted with greater accuracy. Multimeric protein input models, such as Alphabet Inc.'s DeepMind and AlphaFold1/AlphaFold2, are artificial intelligence deep learning programs that can compute complex protein structures such as antigen-antibody interaction. These programs pave the way for combining deep learning technology with energy minimization techniques to improve the accuracy of protein-protein binding affinity computations, as disclosed herein.

The present method combines a physical and statistical algorithm with a deep learning method to compute protein-protein binding affinity with and without 3D protein structures. For example, in the present method, a deep learning model (e.g., Alphabet Inc.'s AlphaFold-Multimer, DeepAB, and ABLooper) is used to compute protein 3D structures for both a protein-protein complex and its constituent parts. A “relax” mode algorithm (e.g., Rosetta Relax, Amber) is used to carry out the task of structural refinement on a plurality of checkpoints of each of the 3D structure models to compute an energy score. Although the protein 3D structure computed using the deep learning model is generally accurate, these models are not physics based and the amino acids and associated atoms may not be feasible physically, e.g., the atoms may clash in physical space. The relax algorithm, e.g., with Amber or Rosetta Relax, is used to fine-tune the 3D structure to ensure it does not clash in physical space. Using the energy score, the relax algorithm minimizes the score function to fine-tune the 3D structure to avoid clashes. The difference between energy scores of the protein-protein complex and the individual protein parts are determined, where the score difference defines a binding affinity score. This 3D structure-based method does not need training data and can be characterized as unsupervised learning. Further, the binding affinity score can be computed without a priori knowledge of 3D structures (without the need for supervised learning).

In an example, a pipeline for computing affinity of flexible complex proteins disclosed herein comprises:
using a deep learning model (e.g., Alphabet, Inc.'s AlphaFold2; Deep AB; or the ABLooper deep-learning based complementarity-determining region (CDR) loop structure prediction tool) to compute protein part (e.g., antigen or antibody) 3D structures. An ensemble of the checkpoints or initial random seeds may be used to find the final binding affinity scores for each model. For example, for both part and complex 3D structures, the deep learning model generates 3D structures with different model checkpoints or different initial random seeds. Each structure is a hypothesis in protein conformational space. This conformational space may be sampled find a top predetermined number ‘N’ hypotheses (e.g., the top five (5) hypotheses) with the lowest energy scores. The mean energy of the top ‘N’ hypothesis is considered as the energy for the protein part or protein complex, and defines the lowest energy score for the protein part or protein complex.
using a deep learning model, e.g., Alphabet, Inc.'s AlphaFold-Multimer deep learning Model, to compute the protein-protein complex (Ag+Ab) 3D structure. For example, if the binding site is known, the protein-protein complex that includes the binding site can be selected as the template input.
using a relax algorithm (e.g., the University of Washington-developed Rosetta Relax mode and Score statistics-based platform or the Amber Relax program (https://ambermd.org/)) on both the side chain (R group) and backbone of each amino acid for of the parts and the complex (Ag, Ab, Ag+Ab) to find a low energy score state and to compute an energy score; and
determining a score difference between the protein-protein complex (Ag+Ab) and the sum of the parts (Ag, Ab), wherein the score difference defines a protein-protein binding affinity score.

Advantageously, the present method combines a protein 3D structure computed using deep learning and a relax and score function from an energy minimization algorithm to improve the accuracy of computing binding affinity of protein-protein complexes. The computation of the parts and the protein-protein complex is decoupled and there is no need to find the absolute lowest low energy state. The present method also reduces computation time over classical methods, since the inference of the 3D protein structure can be performed in several minutes versus in silico protein docking techniques, which require several days of compute time using, e.g., the Metropolis Monte Carlo algorithm. Further, the present method can be implemented using unsupervised learning techniques, and thus does not require 3D protein structure training data (although one or several 3D protein structure training examples can be used in the various embodiments).

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described, unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Throughout the following discussion, numerous references will be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network.

As used in the description herein and throughout the claims that follow, when a system, engine, server, device, module, or other computing element is described as being configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computer should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

The focus of the disclosed inventive subject matter is to enable construction or configuration of a computing device to operate on vast quantities of digital data, beyond the capabilities of a human for purposes including computing protein-protein interaction affinity.

One should appreciate that the disclosed techniques provide many advantageous technical effects including improving the scope, accuracy, compactness, efficiency, and speed of computing protein-protein interaction affinity. It should also be appreciated that the following specification is not intended as an extensive overview, and as such, concepts may be simplified in the interests of clarity and brevity.

In addition to the terms above, the following technical terms are used throughout the specification and claims.

Amino acids are called residues when two or more amino acids bond with each other.

Protein folding is the physical process where a protein chain is translated into its native three-dimensional structure, typically a “folded” conformation, by which the protein becomes biologically functional.

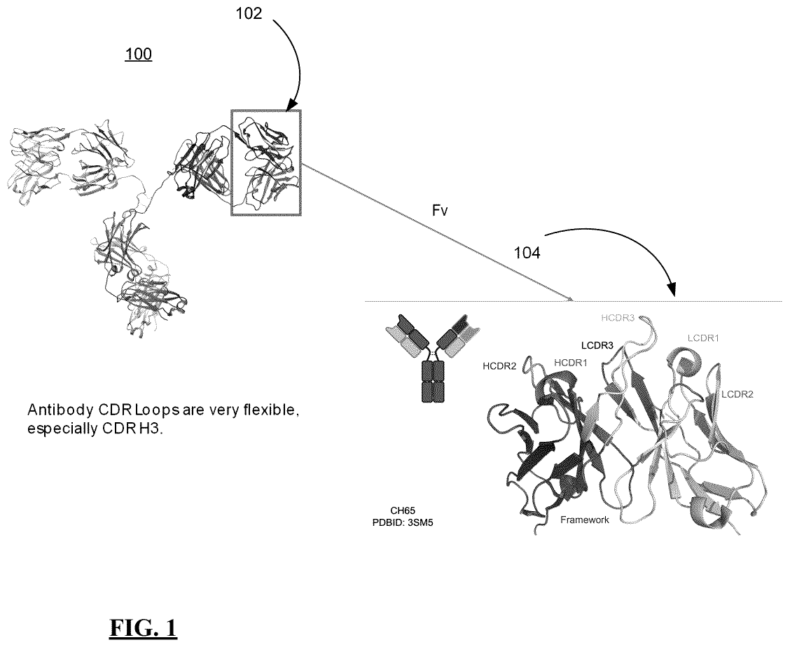

FIG. 1 illustrates a 3D visual representation of protein complementary-determining region (CDR) loops in accordance with an embodiment. In FIG. 1, a protein complementary-determining region (CDR) 100 is shown. A CDR 100 is a variable sequence of amino acids that folds into loops 102 capable of binding to an antigenic amino acid sequence, also known as an epitope. CDRs, framework regions, and residues present in the variable domain play a significant role in binding efficiency and/or specificity of protein-protein (e.g., antigen-antibody) interaction. For example, the human immune system uses two main types of antigen receptors: T-cell receptors (TCRs) and antibodies. While both proteins share a globally similar 8-sandwich domains consisting of 80 to 350 amino acids, TCRs are specialized to recognize peptide antigens in the binding groove of the major histocompatibility complex, while antibodies can bind to a range of molecules. For both proteins, the main determinants of target recognition are their CDR loops that are a certain number of residues in length. Certain CDRs adopt a limited number of backbone conformations, known as the “canonical classes”; but the remaining CDR loops (β3 in TCRs and H3 in antibodies) are very flexible 3D structures that can form complex protein-protein interfaces. For example, CDRH3 104 is a 3D visual representation of a flexible CDRH3 loop. The deep learning-based protein-protein interaction pipeline described herein is operable to generate 3D structure hypotheses related to the interaction residue pairs in protein complex interfaces by combining deep learning models with physics and statistic-based score minimization algorithms.

### Physical and Statistic-Based Score Minimization Algorithms

A score function, e.g., the Rosetta score function, has physical and statistical terms. The total scores are calculated as a weighted sum of individual energy terms, where lower scores indicate more stable structures. The Rosetta score function algorithm generally includes the following: building a 3D protein structure model; relaxing and aligning components to refine/fine-tune the model; and a loop of: selecting a starting conformation with a Metropolis Monte Carlo algorithm; minimizing the score function by selecting and minimizing backbone and side-chain angles (fast Relax); generating a large number of “decoys” (candidate structures); and selecting a decoy with the lowest energy score (Rosetta energy).

A limitation of the energy minimization approach is that it requires many samplings to find the lowest energy state, and the 3D structure and the protein part lowest states are needed to compute docking of the complex, which is a two-stage protocol—the first stage being where aggressive sampling is done, and the second stage where smaller movements take place (in full atom mode). Oftentimes, accuracy is relatively low due to the difficulty to predict the protein 3D structure with the energy minimization method.

### The Novel Deep Learning/Physical and Statistic-Based Approach

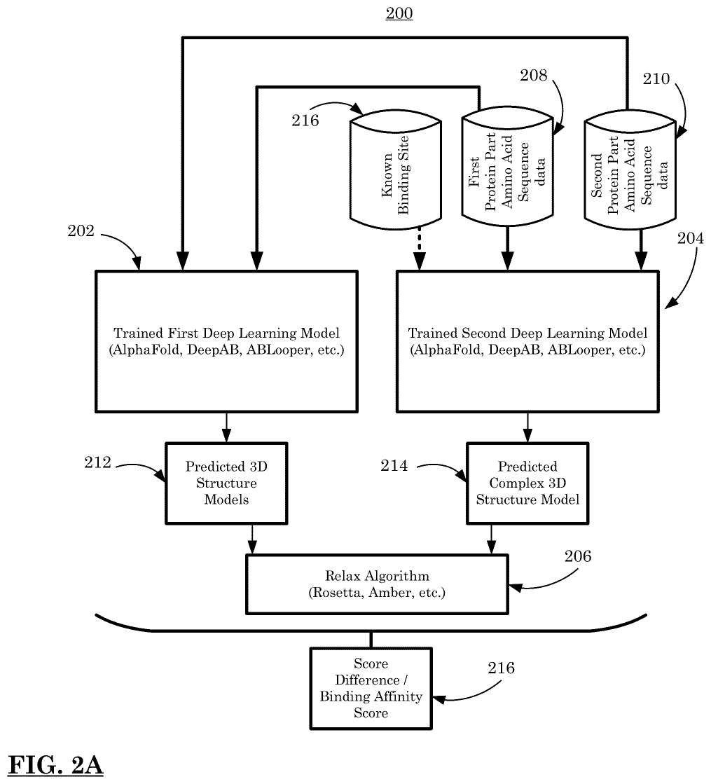

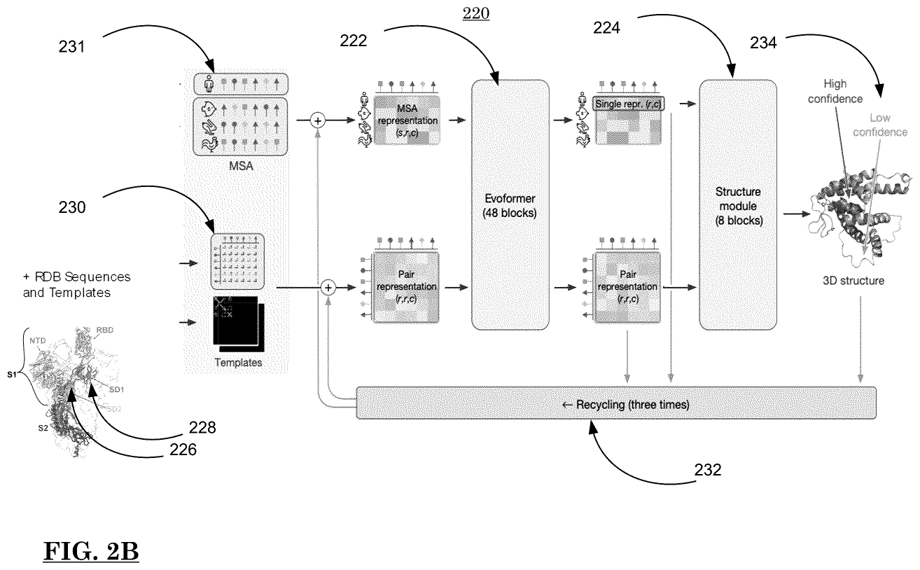

In the embodiments herein, the physical and statistical algorithm is combined with a deep learning method (trained with or without 3D protein structures) to form a unique data pipeline for computing and, in some embodiments, controlling the binding affinity of flexible complex proteins. The protein 3D structures are computed using a deep learning method. But since these models are not physics based, e.g., the amino acids and associated atoms may not be physically feasible, a physical/statistical relax algorithm, e.g., Amber or Rosetta Relax, is used to fine-tune the 3D structures. The relax algorithm ensures the predicted 3D structures do not clash in physical space, and using the energy score, minimizes a score function to fine-tune the 3D structures to avoid clashes. Factors considered for the novel approach include that Rosetta Relax can be run efficiently (with respect to computational cost) due to the relatively simple algorithm, and recent releases of multimer models, e.g., AlphaFold1/AlphaFold2, allow for accurately computing complex 3D protein structures. For example, the combined capabilities of current models, e.g., AlphaFold1/AlphaFold2, DeepAB, and/or ABLooper, can be used to predict the 3D structure of (Ag, Ab, Ag+Ab) with sufficient accuracy to compute affinity of flexible complex proteins and find the interaction residue pairs in the complex protein interfaces. FIG. 2A illustrates a block diagram of a data pipeline for computing protein-protein interaction affinity in accordance with an embodiment. The pipeline 200 of the novel approach comprises a trained first deep learning model 202, a trained second deep learning model 204, and a relax algorithm 206. The trained first deep learning model (e.g., Alphabet, Inc.'s AlphaFold2; Deep AB; or the ABLooper deep-learning based CDR loop structure prediction tool) obtains amino acid sequence data corresponding to a first protein part 208 and a second protein part 210 to compute protein part (e.g., antigen or antibody) 3D structures 212. The trained second deep learning model 204 obtains amino acid sequence data corresponding to the first protein part 208 and the second protein part 210, and computes the protein-protein complex (Ag+Ab) 3D structure 214. An ensemble of checkpoints or initial random seeds may be used to find the final binding affinity scores for each 3D structure model. For example, for both protein part and complex 3D structures, the first/second deep learning model generates 3D structures with different model checkpoints or different initial random seeds. Each structure is a hypothesis in protein conformational space. This conformational space may be sampled find the top ‘N’ hypotheses (e.g., the top five (5) hypotheses) with the lowest energy scores. The mean energy of these top ‘N’ hypotheses is the energy for the protein part or protein complex. This mean energy is called lowest energy score for the protein part or protein complex.

In some embodiments, if the binding site is known, the second deep learning model 204 can select the protein-protein complex that includes the known binding site 216 as the template input.

The relax algorithm 206 (e.g., the University of Washington-developed Rosetta Relax mode and Score statistics-based platform or the Amber Relax program (https://ambermd.org/)) can be used on both the side chain (R group) and backbone of each amino acid for the parts 212 and the complex 214 (Ag, Ab, Ag+Ab) to find a low energy score state and to compute an energy score. The physical/statistical relax algorithm fine-tunes the 3D structures and ensures the predicted 3D structures do not clash in physical space. The energy score minimizes a score function to fine-tune the 3D structures to avoid clashes. The score difference 216 between the protein-protein complex (Ag+Ab) and the sum of the parts (Ag, Ab) can then be determined, where the score difference defines a protein-protein binding affinity score.

FIG. 2B illustrates an example deep learning model for computing protein-protein interaction affinity in accordance with an embodiment. Deep learning model 220 illustrates a design used by AlphaFold 2 comprising a system of sub-networks coupled together into a single end-to-end model, based on pattern recognition, which is trained in an integrated way as a single integrated structure. Two modules, evoformer module 222 and structure prediction module 224, are used to progressively refine a vector of information for each relationship (i.e., “edge”) between an amino acid residue of the protein 226 and another amino acid residue 228 (these relationships are represented by array 230 as shown in green); and between each amino acid position and each different sequences in the input sequence alignment (these relationships are represented by array 230 as shown in red). Internally, these refinement transformations contain layers that have the effect of bringing relevant data together and filtering out irrelevant data (the “attention mechanism”) for these relationships, in a context-dependent way, learned from training data, e.g., Multiple Sequence Alignment (MSA) data 231 comprising an alignment of three or more biological sequences (protein or nucleic acid) of similar length. These transformations are iterated 232, the updated information output by one step becoming the input of the next, with the sharpened residue/residue information feeding into the update of the residue/sequence information, and then the improved residue/sequence information feeding into the update of the residue/residue information. The output of these iterations then informs the structure prediction module 224, which is itself then iterated in order to generate a final 3D structure prediction 234.

While several specific deep learning models, e.g., AlphaFold1/AlphaFold2, DeepAB, ABLooper, etc. and relax algorithms are described as be utilized to perform the various inventive steps herein, one skilled in the art will appreciate that the models/algorithms are merely exemplary and not limiting. Various other deep learning models and algorithms, including variations deep learning models and algorithms either currently available or available in the future, may be suitable for carrying out the various embodiments.

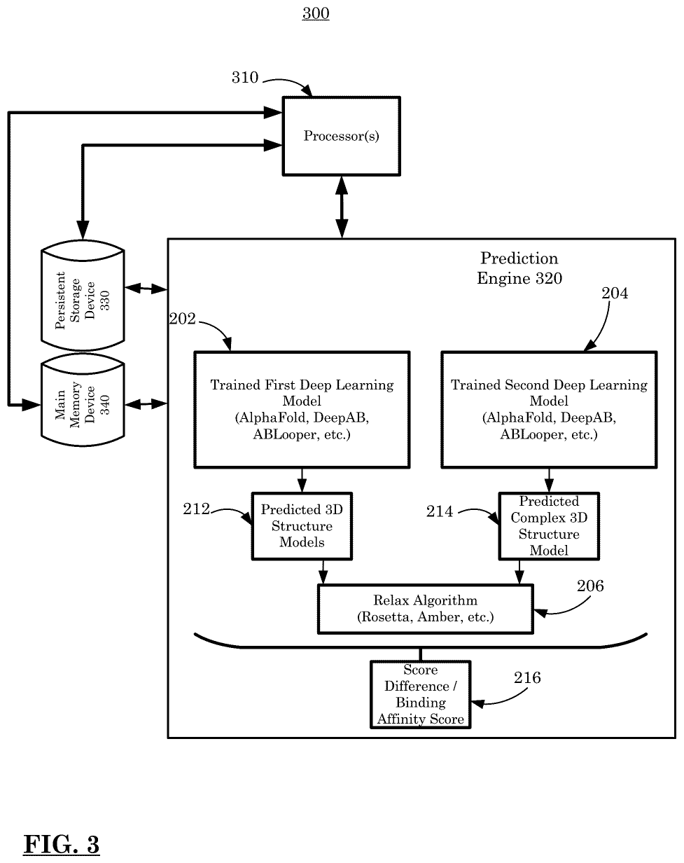

FIG. 3 illustrates a block diagram of a system for computing protein-protein interaction affinity in accordance with an embodiment. System 300 comprises one or more processors 310, a prediction engine 320, a persistent storage device 330, and a main memory device 340. For example, elements for computing protein-protein interaction affinity may include at least one memory (e.g., persistent storage device 330, and main memory device 340) having computer-readable instructions stored thereon which, when executed by the at least one processor 310 coupled to the at least one memory, cause the at least one processor to obtain, from an amino acid sequence database (e.g., persistent storage device 330, and main memory device 340), amino acid sequence data corresponding to a first protein part and a second protein part. For example, the amino acid sequence data corresponding to the first protein part and the second protein part may comprise FASTA format sequence data. As discussed above, the first protein part and the second protein part may each comprise flexible complementary-determining region (CDR) loop structures, e.g., where the first protein part comprises an antigen (Ag) and the second protein part comprises an antibody (Ab).

The at least one processor 310, coupled with and/or operating as prediction engine 320, is further caused to feed the amino acid sequence data corresponding to the first protein part and the second protein part, respectively, into a trained first deep learning model, e.g., trained first deep learning model 202, where the trained first deep learning model is trained to predict a 3D structure model based on a first input of amino acid sequence data corresponding to a protein part. For example, in some embodiments, the trained first deep learning model may generate 3D structure hypotheses using checkpoints or a random seed. The at least one processor 310 is further caused to obtain 3D structure models of the first protein part and the second protein part predicted by the trained first deep learning model.

The at least one processor 310, coupled with and/or operating as prediction engine 320, is further caused to feed the amino acid sequence data corresponding to the first protein part and the second protein part into a trained second deep learning model, e.g., second prediction model 204, where the trained second deep learning model is trained to predict a 3D structure model of a protein-protein complex based on a second input of amino acid sequence data corresponding to protein-protein complex parts. Further, the second deep learning model and the first deep learning model may be the same deep learning model (e.g., one or more of AlphaFold2; DeepAB; ABLooper, or the like). In some embodiments, the protein-protein complex may comprise a known binding site complex, and feeding the amino acid sequence data corresponding to the first protein part and the second protein part into the trained second deep learning model may comprise feeding a third input comprising the known binding site complex into the trained second deep learning model. For example, the known binding site complex may comprise a mutation of the amino acid sequence data corresponding to a first protein part and a second protein part.

The at least one processor 310 is further caused to obtain a 3D structure model of the protein-protein complex comprising the first protein part and the second protein part predicted by the trained second deep learning model.

The at least one processor 310 is further caused to determine a low energy score state for the 3D structure models of each of the first protein part, the second protein part, and the protein-protein complex. In an embodiment, the at least one processor 310/prediction engine 320 may be further caused to use a relax algorithm, e.g., relax algorithm 206, to determine the low energy score state for the 3D structure models of each of the first protein part, the second protein part, and the protein-protein complex. The relax algorithm may be applied to amino acid side chain and backbone 3D structure models of each of the first protein part, the second protein part, and the protein-protein complex to fine tune the 3D structures (e.g., to reconcile the 3D structures within physical space). For example, the relax algorithm may comprise, e.g., at least one of Rosetta Relax or Amber Relax.

The at least one processor 310 is further caused to generate, based on the low energy score states, an energy score for the 3D structure models of each of the first protein part, the second protein part, and the protein-protein complex; and determine a score difference between the energy score for the 3D structure model of the protein-protein complex and a sum of the energy scores for the 3D structure models of first protein part and the second protein part, wherein the score difference defines a binding affinity score. For example, the energy scores for the 3D structure models of each of the first protein part, the second protein part, and the protein-protein complex are generated using a Rosetta Relax score function.

It should be noted that the elements in FIG. 3, and the various functions attributed to each of the elements, while exemplary, are described as such solely for the purposes of ease of understanding. One skilled in the art will appreciate that one or more of the functions ascribed to the various elements may be performed by any one of the other elements, and/or by an element (not shown) configured to perform a combination of the various functions. Therefore, it should be noted that any language directed to a processor(s) 310, a prediction engine 320, a persistent storage device 330 and a main memory device 340 should be read to include any suitable combination of computing devices, including servers, interfaces, systems, databases, agents, peers, engines, controllers, modules, or other types of computing devices operating individually or collectively to perform the functions ascribed to the various elements. Further, one skilled in the art will appreciate that one or more of the functions of the system of FIG. 3 described herein may be performed within the context of a client-server relationship, such as by one or more servers, one or more client devices (e.g., one or more user devices) and/or by a combination of one or more servers and client devices.

While the system illustrated in FIG. 3 is exemplary for implementing the embodiments herein, one skilled in the art will further appreciate that various other systems (e.g., client-server-based systems) and additions (such as deep learning attention mechanisms) may be utilized. As such, system 300 should not be construed as being strictly limited to the embodiments described herein.

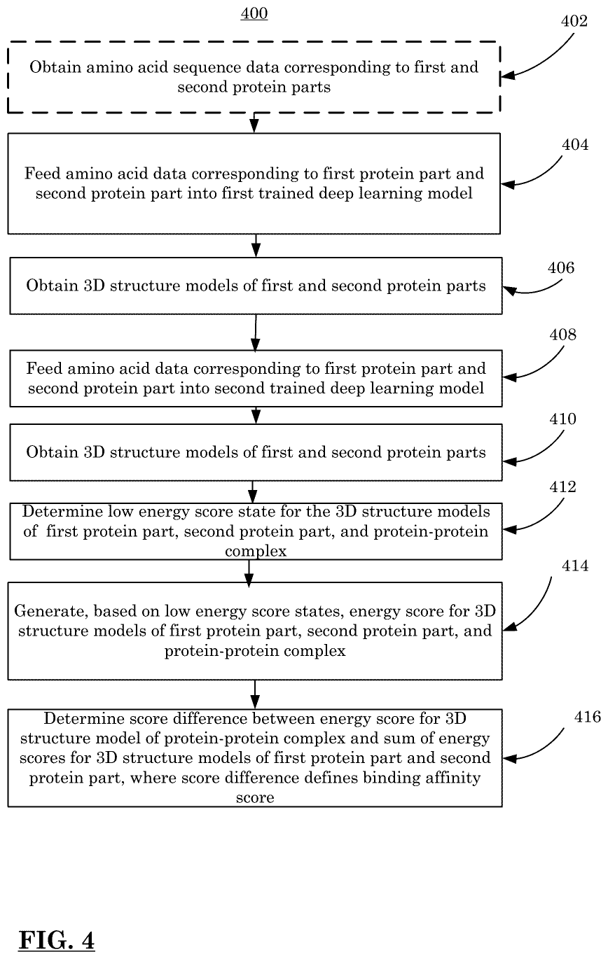

FIG. 4 illustrates a flow diagram of example operations for computing protein-protein interaction affinity in accordance with an embodiment. In flow diagram 400, amino acid sequence data corresponding to a first protein part and a second protein part are obtained, e.g., from an amino acid sequence database, at step 402. At step 404, the amino acid sequence data corresponding to the first protein part and the second protein part, respectively, is feed into a trained first deep learning model, where the trained first deep learning model is trained to predict a 3D structure model based on a first input of amino acid sequence data corresponding to a protein part. At step 406, 3D structure models of the first protein part and the second protein part predicted by the trained first deep learning model are obtained. At step 408, the amino acid sequence data corresponding to the first protein part and the second protein part is feed into a trained second deep learning model, where the trained second deep learning model is trained to predict a 3D structure model of a protein-protein complex based on a second input of amino acid sequence data corresponding to protein-protein complex parts. At step 410, a 3D structure model of the protein-protein complex comprising the first protein part and the second protein part predicted by the trained second deep learning model is obtained. In various embodiments, an ensemble of checkpoints or initial random seeds may be used to find the final binding affinity scores for each 3D structure model. For example, for both protein part and complex 3D structures, the deep learning model can generate 3D structures with different model checkpoints or different initial random seeds. Each structure is a hypothesis in protein conformational space.

At step 412, a low energy score state is determined for the 3D structure models of each of the first protein part, the second protein part, and the protein-protein complex. For example, the protein conformational space may be sampled to find a top predetermined number ‘N’ of hypotheses (e.g., the top five (5) hypotheses) with the lowest energy scores. The mean energy of these top ‘N’ hypotheses is the energy for the protein part or protein complex, which is called lowest energy score for the protein part or protein complex. At step 414, based on the low energy score states, an energy score is generated for the 3D structure models of each of the first protein part, the second protein part, and the protein-protein complex, and a score difference is determined between the energy score for the 3D structure model of the protein-protein complex and a sum of the energy scores for the 3D structure models of the first protein part and the second protein part at step 416, where the score difference defines a binding affinity score.

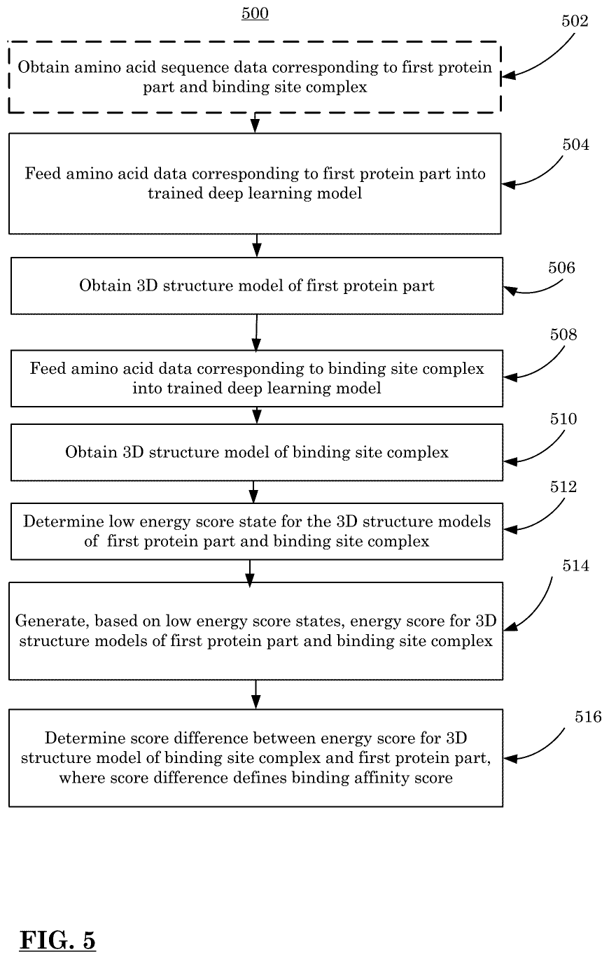

FIG. 5 illustrates a flow diagram of example operations for computing protein-protein interaction affinity in accordance with an embodiment. In flow diagram 500, amino acid sequence data corresponding to first protein part and a binding site complex are known. Therefore, at step 502, amino acid sequence data corresponding to first protein part and a binding site complex are obtained, e.g., from an amino acid sequence database, where the binding site complex corresponds to a known binding site between the first protein part and a second protein part. At step 504, the amino acid sequence data corresponding to the first protein part is feed into a trained deep learning model, where the trained deep learning model is trained to predict a 3D structure model based on a first input of amino acid sequence data corresponding to a protein part or a protein-protein complex. At step 506, a 3D structure model of the first protein part predicted by the trained deep learning model is obtained. At step 508, the amino acid sequence data corresponding to the binding site complex is feed into the trained deep learning model. At step 510, a 3D structure model of the binding site complex predicted by the trained deep learning model is obtained. In various embodiments, an ensemble of checkpoints or initial random seeds may be used to find the final binding affinity scores for each 3D structure model. For example, for both protein part and complex 3D structures, the deep learning model can generate 3D structures with different model checkpoints or different initial random seeds. Each structure is a hypothesis in protein conformational space.

At step 512, a low energy score state is determined for the 3D structure models of each of the first protein part and the binding site complex. For example, the protein conformational space may be sampled to find a top predetermined number ‘N’ of hypotheses (e.g., the top five (5) hypotheses) with the lowest energy scores. The mean energy of these top ‘N’ hypotheses is the energy for the protein part or protein complex, which is called lowest energy score for the protein part or protein complex. At step 514, based on the low energy score states, an energy score is generated for the 3D structure models of each of the first protein part and the binding site complex, and a score difference is determined between the energy score for the 3D structure model of the binding site complex and the energy score for the 3D structure model of first protein part at step 516, where the score difference defines a binding affinity score.

### Controlling Binding Affinity

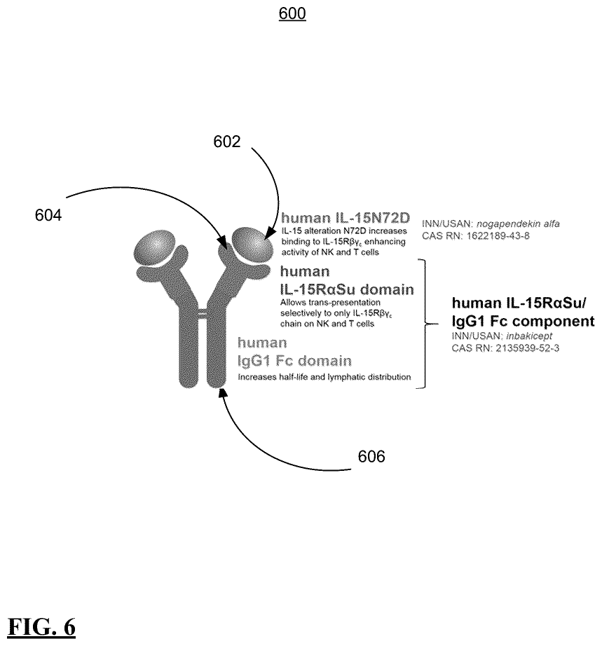

FIG. 6 illustrates a visual 2D representation of a protein complex in accordance with an embodiment. In representation 600, an N803-IL15Rβγ protein complex is shown. Cytokines are a broad and loose category of small proteins important in cell signaling. The cytokine interleukin-15 (IL-15) plays a crucial role in the immune system by affecting the development, maintenance, and function of the natural killer (NK) and T cells. N-803 is a novel IL-15 superagonist complex consisting of an IL-15 mutant (IL-15N72D) 602 bound to an IL-15 receptor a 604/IgG1 Fc 606 fusion protein. Its mechanism of action is direct specific stimulation of CD8+ T cells and NK cells through beta gamma (βγ) T-cell receptor binding (not a) while avoiding T-reg stimulation. Advantageously, N-803 has improved pharmacokinetic properties, longer persistence in lymphoid tissues and enhanced anti-tumor activity compared to native, non-complexed IL-15 in vivo. While N-803/IL-15 binding affinity is an exemplary application of the embodiments herein, one skilled in the art will understand that while the examples are described with respect to N-803/IL-15, the concepts presented apply to any protein-protein combination. Therefore, the examples presented, while illustrative, should not be construed as limiting the various embodiments.

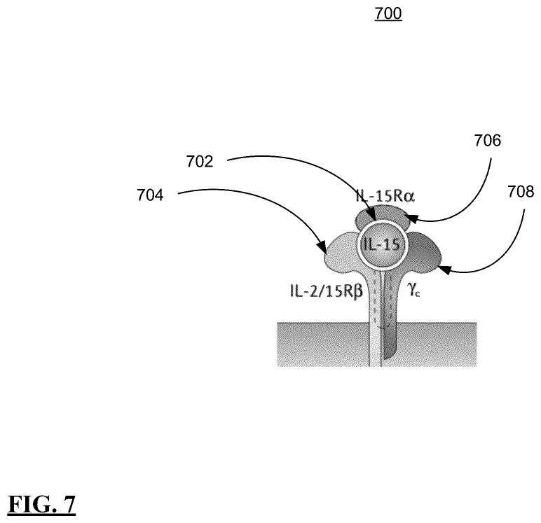

FIG. 7 illustrates a visual 2D representation of an N803-IL15Rβγ protein complex in accordance with an embodiment. In representation 700, the various structures of IL-15: IL-15 702, IL-2/15Rβ 704, IL-15Rα 706, and IL-15Rγ 708 are shown. As noted above, the understanding of interactions between these structures and other protein structures can be crucial to many scientifically useful endeavors, including controlling binding affinity between protein-protein complexes.

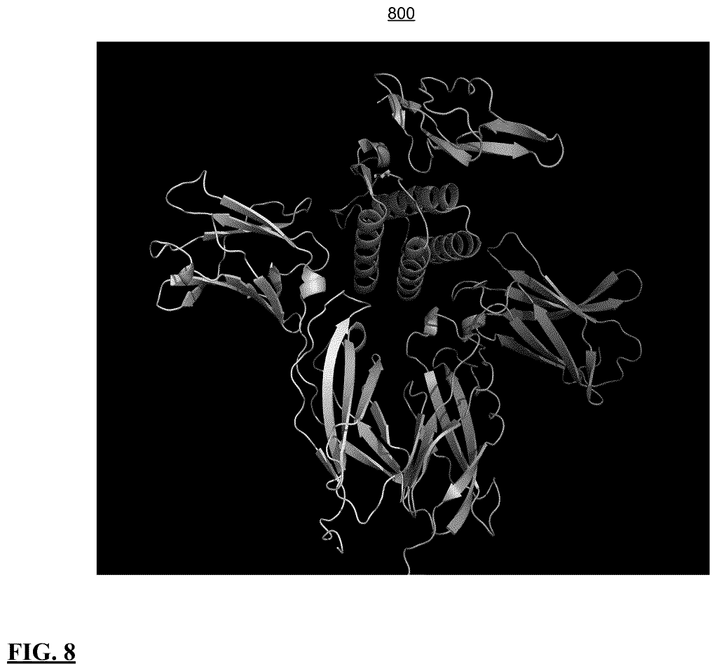

FIG. 8 illustrates a visual 3D representation of an N803-IL15Rβγ protein complex in accordance with an embodiment. In representation 800, IL-15 702, IL-2/15Rβ 704, IL-15Rα 706, and IL-15Rγ 708 are shown in 3D, where the loop structures of the binding site complexes are represented. Using the concepts disclosed herein, the hypothesized 3D models can be used to find and select interaction residue pairs in the protein complex interfaces, and control (increase, decrease, or otherwise tailor) protein-protein binding affinity.

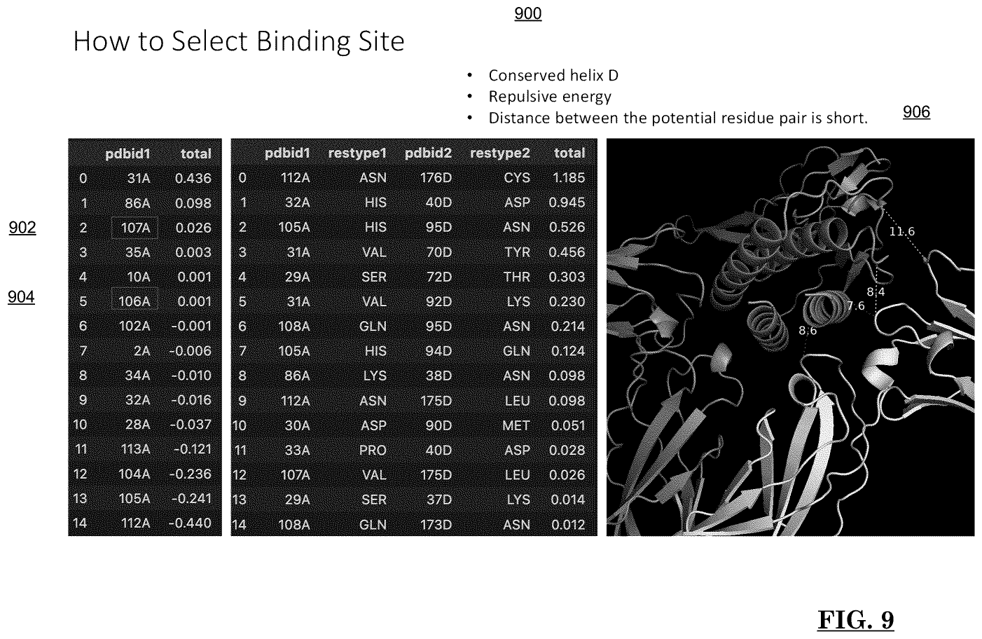

FIG. 9 illustrates a graphical representation related to increasing binding affinity between IL15/IL15Ra and IL15Rb/IL15Rg in accordance with an embodiment. In some instances, e.g., to increase the absorption rate of medications or other treatments, it may be advantageous to increase binding affinity between protein sequences. As shown in representation 900, at least one interaction of residue pairs in interfaces between the first and second protein sequences may be selected based on a binding affinity score. For example, the selection of residue pairs, e.g., residue pairs 107A at 904 and 106A at 904, may be based on at least one of the following: the at least one interaction comprising a conserved helix (relatively stable) structure, a repulsive energy between the potential residue pairs, or a distance between the potential residue pairs. For example, a binding site with too great a distance between residue pairs, or that occurs outside of the relatively stable helix structure, may not be advantageous for controlling binding affinity. Conversely, a good candidate binding site may have a relatively short distance between residue pairs and occur within a stable helix structure.

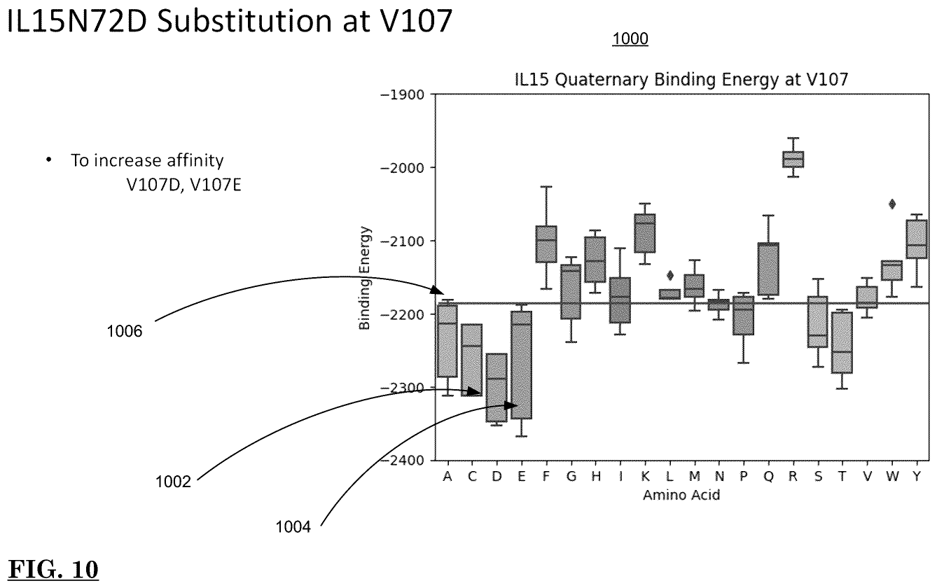

FIG. 10 illustrates a graphical representation related to increasing binding affinity between IL15/IL15Ra and IL15Rb/IL15Rg in accordance with an embodiment. Representation 1000 illustrates the IL15 quaternary binding energy at amino acid V107, represented by positions A-Y, where binding affinity scores have been calculated using the methods described herein. For example, amino acids V107D at 1002 and V107E at 1004 may be selected for substitution based on their relatively low binding energies (with respect to a binding energy mean 1006) to increase binding affinity between IL15/IL15Ra and IL15Rb/IL15Rg.

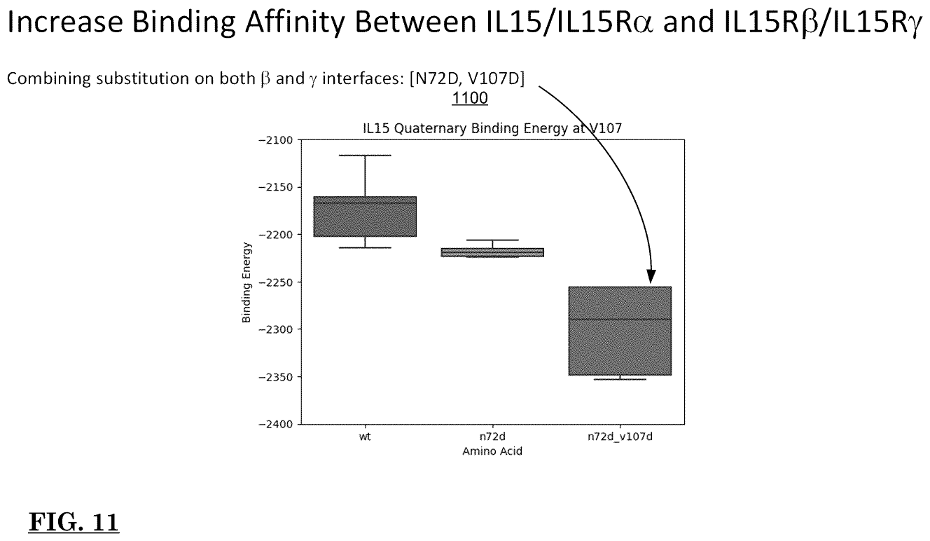

FIG. 11 illustrates a graphical representation related to increasing binding affinity between IL15/IL15Ra and IL15Rb/IL15Rg in accordance with an embodiment. As illustrated in representation 1100, facilitating a combined substitution on both beta and gamma interfaces [N72D, V107D] increases the binding affinity (lowers the overall binding energy) between IL15/IL15Ra and IL15Rb/IL15Rg.

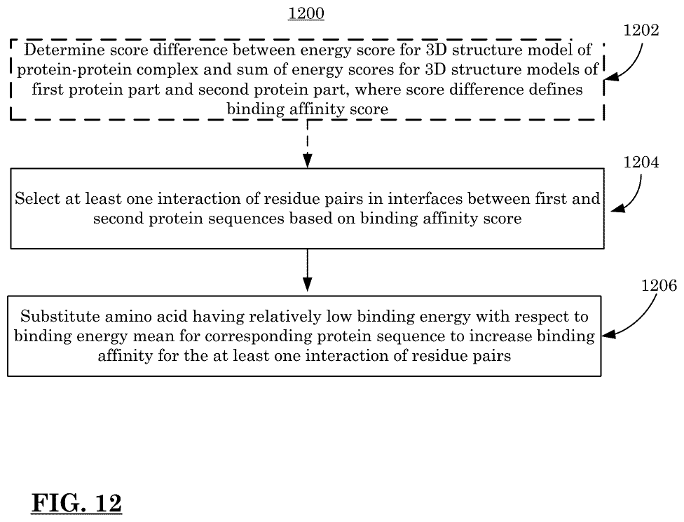

FIG. 12 illustrates a flow diagram of example operations for increasing protein-protein binding affinity in accordance with an embodiment. In flow diagram 1200, at step 1202 and as described in the various embodiment herein, a score difference is determined between an energy score for a 3D structure model of a protein-protein complex and a sum of energy scores for 3D structure models of a first protein part and a second protein part, where the score difference defines a binding affinity score. At least one interaction of residue pairs in interfaces between the first and second protein sequences is selected based on the binding affinity score at step 1204. For example, the selection of the at least one interaction of residue pairs may be based on at least one of the following: the at least one interaction comprising a conserved helix structure, a repulsive energy between the potential residue pairs, or a distance between the potential residue pairs. At step 1206, at least one amino acid of the first or second protein sequences is substituted (i.e., a substitution is facilitated) to control a binding affinity for the at least one interaction of residue pairs. In an embodiment, at least one substitution of the at least one amino acid comprises substituting an amino acid having a relatively low binding energy with respect to a binding energy mean for a corresponding protein sequence to increase the binding affinity for the at least one interaction of residue pairs.

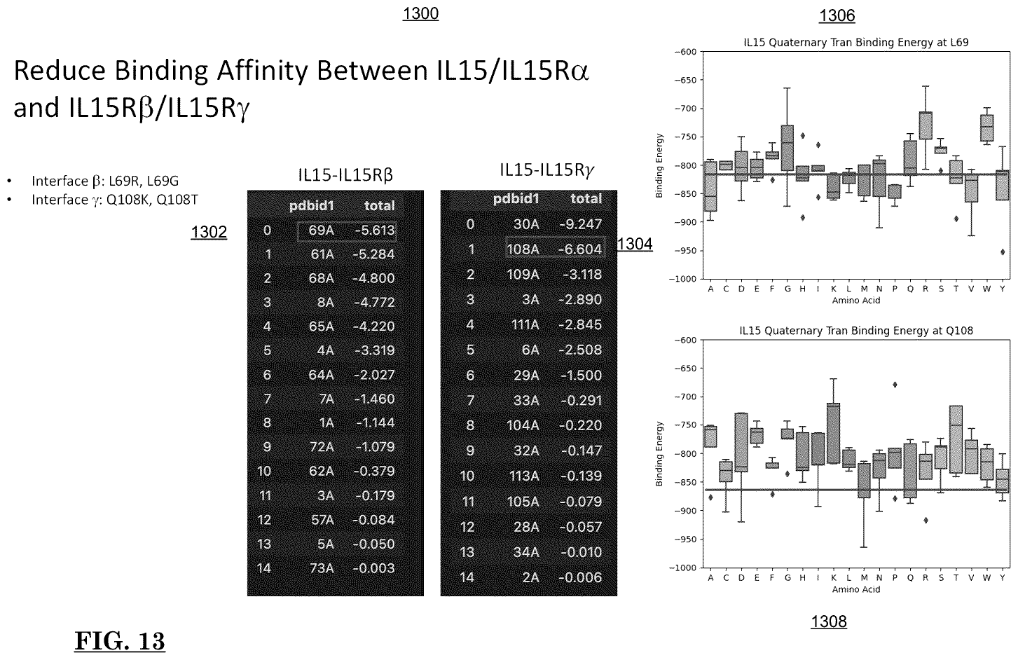

FIG. 13 illustrates a graphical representation related to decreasing binding affinity between IL15/IL15Ra and IL15Rb/IL15Rg in accordance with an embodiment. In some instances, e.g., for quick-absorbing or delayed release medications or other treatments, it may be advantageous to reduce binding affinity between protein sequences. With the techniques described herein, binding affinity data for interacting residue pairs can be used to control the binding affinity of selected interfaces. Representation 1300 illustrates binding affinity scores for IL15-IL15B and IL15-IL15γ interfaces. For example, based on having a relatively high binding energy with respect to a binding energy mean for their respective protein sequences, residue pairs L69 and Q108 (1302 and 1304, respectively) may be selected to decrease the binding affinity for the IL15a-IL15β and IL15a-IL15γ interfaces. As shown in the graphical representations 1306 and 1308, respectively, B interface candidates L69R, L69G and γ interface candidates Q108K and Q108T have the highest binding energy top ranges with respect to their respective binding energy means. Therefore, these candidates may be selected for substitution to reduce binding affinity.

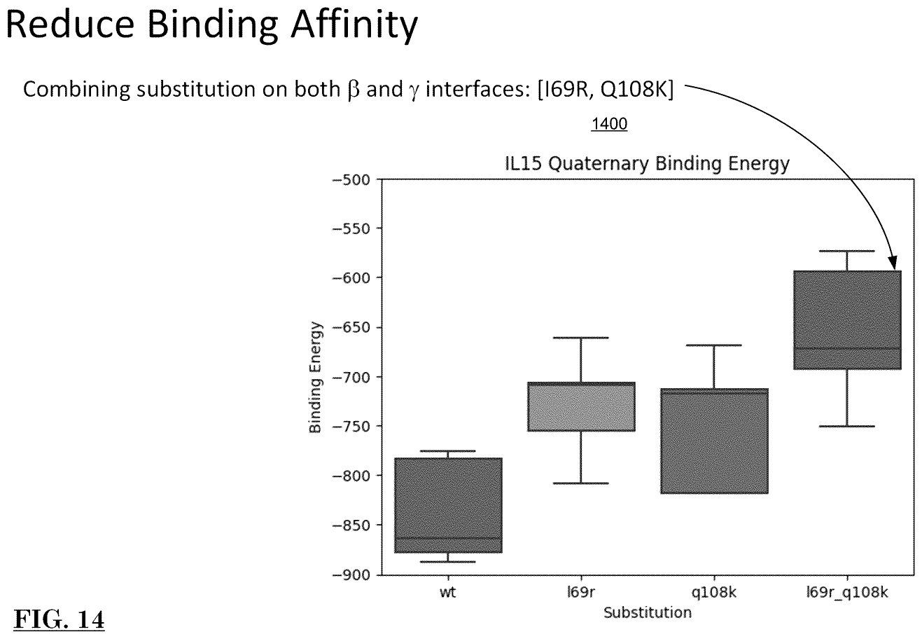

FIG. 14 illustrates a graphical representation related to decreasing binding affinity between IL15/IL15Ra and IL15Rb/IL15Rg in accordance with an embodiment. As illustrated in representation 1400, a combined substitution on both B and γ interfaces [I69R, Q108K] reduces the binding affinity between IL15/IL15Ra and IL15Rb/IL15Rg. Thus, facilitating substitution of I69R and Q108K, which have a relatively high binding energy with respect to a binding energy mean for the corresponding protein sequence, decreases the binding affinity for the interaction of residue pairs.

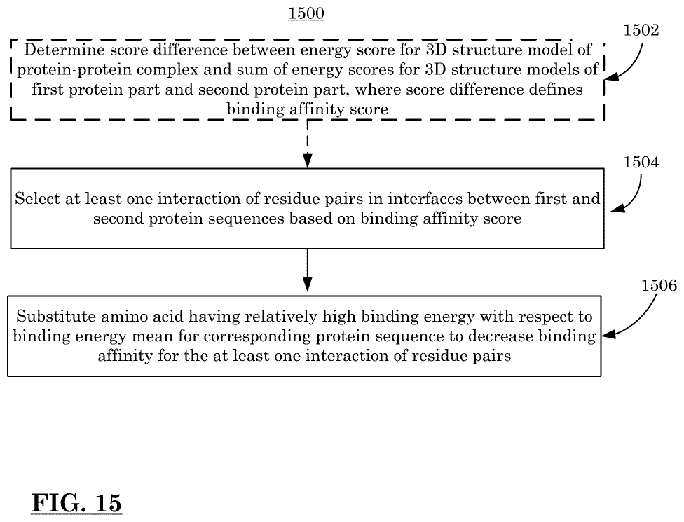

FIG. 15 illustrates a flow diagram of example operations for decreasing protein-protein binding affinity in accordance with an embodiment. In flow diagram 1500, at 1502 and as described in the various embodiment herein, a score difference is determined between an energy score for a 3D structure model of a protein-protein complex and a sum of energy scores for 3D structure models of a first protein part and a second protein part, where the score difference defines a binding affinity score. At least one interaction of residue pairs in interfaces between the first and second protein sequences is selected based on the binding affinity score at step 1504. For example, the selection of the at least one interaction of residue pairs may be based on at least one of the following: the at least one interaction comprising a conserved helix structure, a repulsive energy between the potential residue pairs, or a distance between the potential residue pairs. At step 1506, at least one amino acid of the first or second protein sequences is substituted to control a binding affinity for the at least one interaction of residue pairs. In an embodiment, substituting the at least one amino acid comprises substituting an amino acid having a relatively high binding energy with respect to a binding energy mean for a corresponding protein sequence to decrease the binding affinity for the at least one interaction of residue pairs.

Thus, in the embodiments herein, amino acid sequence data (e.g., FASTA format sequence data) corresponding to a first protein part and a second protein part, is obtained, e.g., from persistent storage device 330 and/or main memory device 340. For example, the first protein part and a second protein part may comprise flexible complex proteins, e.g., an antigen (Ag) and an antibody (Ab). 3D structure models of the first protein part and the second protein part are generated using a trained first deep learning model, where the trained first deep learning model outputs the 3D structure models of the first protein part and the second protein part based on first inputs comprising the amino acid sequence data corresponding to the first protein part and second inputs comprising the amino acid sequence data corresponding to the second protein part, respectively. The first deep learning model may comprise at least one of the following: AlphaFold2; AlphaFold-Multimer; Deep AB; or ABLooper. For example, the first deep learning model may use an ensemble of checkpoints or initial random seeds for determining the 3D structure models of the first protein part and the second protein part. A 3D structure model of a protein-protein complex comprising the first protein part and the second protein part is generated using a trained second deep learning model, where the trained second deep learning model outputs a 3D structure model of a protein-protein complex comprising the first protein part and the second protein part based on third inputs comprising the amino acid sequence data corresponding to the first protein part and the second protein part. The second deep learning model may comprise at least one of the following: AlphaFold2; AlphaFold-Multimer; Deep AB; or ABLooper, and may be the same as the first deep learning model. A low energy score state is determined for the 3D structure models of each of the first protein part, the second protein part, and the protein-protein complex. A relax algorithm applied to amino acid side chain and backbone 3D structure models of each of the first protein part, second protein part, and protein-protein complex may be used to determine the low energy score state for the 3D structure models of each of the first protein part, the second protein part, and the protein-protein complex. For example, the relax algorithm may comprise at least one of the following: Rosetta Relax or Amber Relax. Based on the low energy score states, an energy score is generated for the 3D structure models of each of the first protein part, the second protein part, and the protein-protein complex; and a score difference is determined between the energy score for the 3D structure model of the protein-protein complex and a sum of the energy scores for the 3D structure models of the first protein part and the second protein part, where the score difference defines a binding affinity score. The binding affinity scores of interaction residue pairs may be stored in either one or both of persistent storage device 330 and main memory device 340. Further, at least one interaction of residue pairs in interfaces between the first and second protein sequences may be selected based on the binding affinity score, and substitution of at least one amino acid of the first or second protein sequences may be facilitated to control a binding affinity for the at least one interaction of residue pairs. The selection of the at least one interaction of residue pairs may be based on at least one of: the at least one interaction comprising a conserved helix structure, a repulsive energy between the potential residue pairs, or a distance between the potential residue pairs.

One skilled in the art will appreciate that the systems, apparatus, and methods described herein may be implemented using a client-server relationship, and that many client-server relationships that are possible for implementing the systems, apparatus, and methods described herein. Examples of client devices can include cellular smartphones, kiosks, personal data assistants, tablets, robots, vehicles, web cameras, or other types of computing devices.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIGS. 4, 5, 12 and 15, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

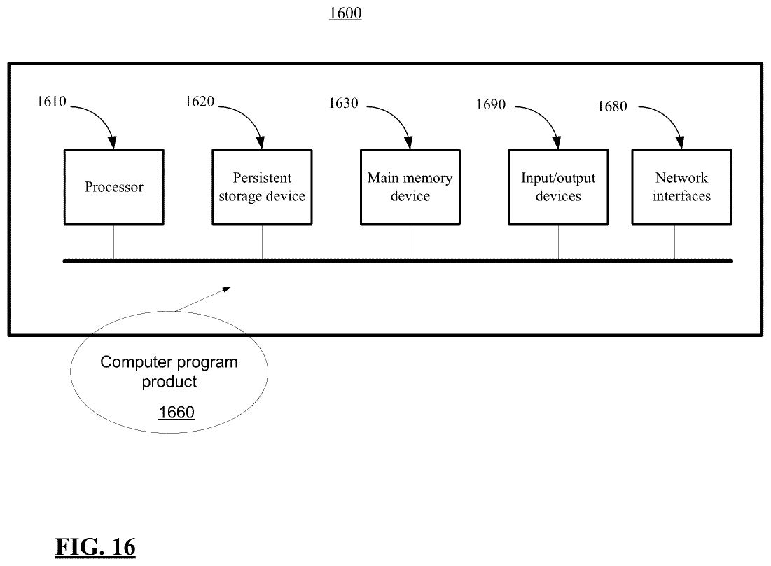

A high-level block diagram of an exemplary apparatus that may be used to implement systems, apparatus and methods described herein is illustrated in FIG. 16. Apparatus 1600 comprises a processor 1610 operatively coupled to a persistent storage device 1620 and a main memory device 1630. Processor 1610 controls the overall operation of apparatus 1600 by executing computer program instructions that define such operations. The computer program instructions may be stored in persistent storage device 1620, or other computer-readable medium, and loaded into main memory device 1630 when execution of the computer program instructions is desired. For example, processor 310 and prediction engine 320 may comprise one or more components of computer 1600. Thus, the method steps of FIGS. 4, 5, 12 and 15 can be defined by computer program instructions stored in main memory device 1630, persistent storage device 2020 and/or computer program product 1660 and controlled by processor 1610 executing the computer program instructions. For example, the computer program instructions can be implemented as computer executable code programmed by one skilled in the art to perform an algorithm defined by one or more of the method steps of FIGS. 4, 5, 12 and 15. Accordingly, by executing the computer program instructions, the processor 1610 executes an algorithm defined by the method steps of FIGS. 4, 5, 12 and 15. Apparatus 1600 also includes one or more network interfaces 1680 for communicating with other devices via a network. Apparatus 1600 may also include one or more input/output devices 1690 that enable user interaction with apparatus 1600 (e.g., display, keyboard, mouse, speakers, buttons, etc.).

Processor 1610 may include both general and special purpose microprocessors and may be the sole processor or one of multiple processors of apparatus 1600. Processor 1610 may comprise one or more central processing units (CPUs), and one or more graphics processing units (GPUs), which, for example, may work separately from and/or multi-task with one or more CPUs to accelerate processing, e.g., for various image processing applications described herein. Processor 1610, persistent storage device 1620, and/or main memory device 1630 may include, be supplemented by, or incorporated in, one or more application-specific integrated circuits (ASICs) and/or one or more field programmable gate arrays (FPGAs).

Persistent storage device 1620 and main memory device 1630 each comprise a tangible non-transitory computer readable storage medium. Persistent storage device 1620, and main memory device 1630, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1690 may include peripherals, such as a printer, scanner, display screen, etc. For example, input/output devices 1690 may include a display device such as a cathode ray tube (CRT), plasma or liquid crystal display (LCD) monitor for displaying information (e.g., a DNA accessibility prediction result) to a user, a keyboard, and a pointing device such as a mouse or a trackball by which the user can provide input to apparatus 1600.

Any or all of the systems and apparatuses discussed herein, including processor 310 and prediction engine 320 may be performed by, and/or incorporated in, an apparatus such as apparatus 1600. Further, apparatus 1600 may utilize one or more neural networks or other deep-learning techniques to perform prediction engine 320 or other systems or apparatuses discussed herein.

One skilled in the art will recognize that an implementation of an actual computer or computer system may have other structures and may contain other components as well, and that FIG. 16 is a high-level representation of some of the components of such a computer for illustrative purposes.

The foregoing specification is to be understood as being in every respect illustrative and exemplary, but not restrictive, and the scope of the invention disclosed herein is not to be determined from the specification, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

We claim:

1. A computerized method for determining protein-protein interaction affinity, comprising:
obtaining, from an amino acid sequence database, amino acid sequence data corresponding to a first protein part and a second protein part;
feeding the amino acid sequence data corresponding to the first protein part and the second protein part, respectively, into a trained first deep learning model, wherein the trained first deep learning model is trained to predict a 3D structure model based on a first input of amino acid sequence data corresponding to a protein part;
obtaining 3D structure models of the first protein part and the second protein part predicted by the trained first deep learning model;
feeding the amino acid sequence data corresponding to the first protein part and the second protein part into a trained second deep learning model, wherein the trained second deep learning model is trained to predict a 3D structure model of a protein-protein complex based on a second input of amino acid sequence data corresponding to protein-protein complex parts;
obtaining a 3D structure model of the protein-protein complex comprising the first protein part and the second protein part predicted by the trained second deep learning model;
determining a low energy score state for the 3D structure models of each of the first protein part, the second protein part, and the protein-protein complex;
generating, based on the low energy score states, an energy score for the 3D structure models of each of the first protein part, the second protein part, and the protein-protein complex; and
determining a score difference between the energy score for the 3D structure model of the protein-protein complex and a sum of the energy scores for the 3D structure models of the first protein part and the second protein part, wherein the score difference defines a binding affinity score.

2. The method of claim 1, wherein the first deep learning model and second deep learning model use an ensemble of different model checkpoints or different initial random seeds to find binding affinity scores for each 3D structure model, wherein, for each of the 3D structural models, a protein conformational space is sampled to find a top predetermined number of hypotheses with the lowest energy scores, and wherein a mean energy of the top predetermined number of hypotheses is defined as the low energy score state for the protein part or protein complex.

3. The method of claim 2, wherein the top predetermined number of hypotheses comprises at least five hypotheses.

4. The method of claim 1, wherein the first deep learning model and the second deep learning model comprise at least one of the following: AlphaFold1; AlphaFold2; AlphaFold-Multimer; Deep AB; or ABLooper.

5. The method of claim 4, wherein the second deep learning model is the first deep learning model.

6. The method of claim 1, further comprising using a relax algorithm to determine the low energy score state for the 3D structure models of each of the first protein part, the second protein part, and the protein-protein complex.

7. The method of claim 6, wherein the relax algorithm is applied to amino acid side chain and backbone 3D structure models of each of the first protein part, the second protein part, and the protein-protein complex.

8. The method of claim 6, wherein the relax algorithm comprises at least one of the following: Rosetta Relax or Amber Relax.

9. The method of claim 8, wherein the energy scores for the 3D structure models of each of the first protein part, the second protein part, and the protein-protein complex are generated using a Rosetta Relax score function.

10. The method of claim 1, wherein the first protein part and the second protein part each comprise flexible complementary-determining region (CDR) loop structures.

11. The method of claim 10, wherein the first protein part comprises an antigen (Ag).

12. The method of claim 11, wherein the second protein part comprises an antibody (Ab).

13. The method of claim 1, wherein the protein-protein complex comprises a known binding site complex, and wherein feeding the amino acid sequence data corresponding to the first protein part and the second protein part into the trained second deep learning model comprises feeding a third input comprising the known binding site complex into the trained second deep learning model.

14. The system of claim 13, wherein the known binding site complex comprises a mutation of the amino acid sequence data corresponding to a first protein part and a second protein part.

15. The method of claim 1, wherein the amino acid sequence data corresponding to a first protein part and a second protein part comprises FASTA format sequence data.

16. The method of claim 1, further comprising:
selecting at least one interaction of residue pairs in interfaces between the first and second protein sequences based on the binding affinity score; and
substituting at least one amino acid of the first or second protein sequences to control a binding affinity for the at least one interaction of residue pairs.

17. The method of claim 16, wherein the selection of the at least one interaction of residue pairs is based on at least one of: the at least one interaction comprising a conserved helix structure, a repulsive energy between the potential residue pairs, or a distance between the potential residue pairs.

18. The method of claim 16, wherein substituting the at least one amino acid comprises substituting an amino acid having a relatively low binding energy with respect to a binding energy mean for a corresponding protein sequence to increase the binding affinity for the at least one interaction of residue pairs.

19. The method of claim 16, wherein substituting the at least one amino acid comprises substituting an amino acid having a relatively high binding energy with respect to a binding energy mean for a corresponding protein sequence to decrease the binding affinity for the at least one interaction of residue pairs.

20. A system comprising:
at least one memory having computer-readable instructions stored thereon which, when executed by at least one processor coupled to the at least one memory, cause the at least one processor to:
obtain, from an amino acid sequence database, amino acid sequence data corresponding to a first protein part and a second protein part;
feed the amino acid sequence data corresponding to the first protein part and the second protein part, respectively, into a trained first deep learning model, wherein the trained first deep learning model is trained to predict a 3D structure model based on a first input of amino acid sequence data corresponding to a protein part;
obtain 3D structure models of the first protein part and the second protein part predicted by the trained first deep learning model;
feed the amino acid sequence data corresponding to the first protein part and the second protein part into a trained second deep learning model, wherein the trained second deep learning model is trained to predict a 3D structure model of a protein-protein complex based on a second input of amino acid sequence data corresponding to protein-protein complex parts;
obtain a 3D structure model of the protein-protein complex comprising the first protein part and the second protein part predicted by the trained second deep learning model;
determine a low energy score state for the 3D structure models of each of the first protein part, the second protein part, and the protein-protein complex;
generate, based on the low energy score states, an energy score for the 3D structure models of each of the first protein part, the second protein part, and the protein-protein complex; and
determine a score difference between the energy score for the 3D structure model of the protein-protein complex and a sum of the energy scores for the 3D structure models of first protein part and the second protein part, wherein the score difference defines a binding affinity score.

21. The system of claim 20, wherein the first deep learning model and second deep learning model use an ensemble of different model checkpoints or different initial random seeds to find binding affinity scores for each 3D structure model, wherein, for each of the 3D structural models, a protein conformational space is sampled to find a top predetermined number of hypotheses with the lowest energy scores, and wherein a mean energy of the top predetermined number of hypotheses is defined as the low energy score state for the protein part or protein complex.

22. The system of claim 21, wherein the top predetermined number of hypotheses comprises at least five hypotheses.

23. The system of claim 20, wherein the first deep learning model and the second deep learning model comprise at least one of the following: AlphaFold2; AlphaFold-Multimer; Deep AB; or ABLooper.

24. The system of claim 23, wherein the second deep learning model is the first deep learning model.

25. The system of claim 20, wherein the at least one processor is further caused to use a relax algorithm to determine the low energy score state for the 3D structure models of each of the first protein part, the second protein part, and the protein-protein complex.

26. The system of claim 25, wherein the relax algorithm is used on amino acid side chain and backbone 3D structure features of each of the first protein part, second protein part, and protein-protein complex.

27. The system of claim 25, wherein the relax algorithm comprises at least one of the following: Rosetta Relax or Amber Relax.

28. The system of claim 27, wherein the energy scores for the 3D structure models of each of the first protein part, the second protein part, and the protein-protein complex are generated using the Rosetta Relax score function.

29. The system of claim 20, wherein the first protein part and the second protein part each comprise flexible complementary-determining region (CDR) loop structures.

30. The system of claim 29, wherein the first protein part comprises an antigen (Ag).

31. The system of claim 30, wherein the second protein part comprises an antibody (Ab).

32. The system of claim 20, wherein the protein-protein complex comprises a known binding site complex, and wherein feeding the amino acid sequence data corresponding to the first protein part and the second protein part into the trained second deep learning model comprises feeding a third input comprising the known binding site complex into the trained second deep learning model.

33. The system of claim 32, wherein the known binding site complex comprises a mutation of the amino acid sequence data corresponding to a first protein part and a second protein part.

34. The system of claim 20, wherein the amino acid sequence data corresponding to a first protein part and a second protein part comprises FASTA format sequence data.

35. The system of claim 20, wherein the at least one processor is further caused to:
select at least one interaction of residue pairs in interfaces between the first and second protein sequences based on the binding affinity score; and
substitute at least one amino acid of the first or second protein sequences to control a binding affinity for the at least one interaction of residue pairs.

36. The system of claim 35, wherein the selection of the at least one interaction of residue pairs is based on at least one of: the at least one interaction comprising a conserved helix structure, a repulsive energy between the potential residue pairs, or a distance between the potential residue pairs.

37. The system of claim 35, wherein substituting the at least one amino acid comprises substituting an amino acid having a relatively low binding energy with respect to a binding energy mean for a corresponding protein sequence to increase the binding affinity for the at least one interaction of residue pairs.

38. The system of claim 35, wherein substituting the at least one amino acid comprises substituting an amino acid having a relatively high binding energy with respect to a binding energy mean for a corresponding protein sequence to decrease the binding affinity for the at least one interaction of residue pairs.

39. A computer program product having computer-readable instructions stored thereon, which, when executed by at least one processor, cause the at least one processor to:
obtain, from an amino acid sequence database, amino acid sequence data corresponding to a first protein part and a second protein part;
feed the amino acid sequence data corresponding to the first protein part and the second protein part, respectively, into a trained first deep learning model, wherein the trained first deep learning model is trained to predict a 3D structure model based on a first of input amino acid sequence data corresponding to a protein part;
obtain 3D structure models of the first protein part and the second protein part predicted by the trained first deep learning model;
feed the amino acid sequence data corresponding to the first protein part and the second protein part into a trained second deep learning model, wherein the trained second deep learning model is trained to predict a 3D structure model of a protein-protein complex based on a second input of amino acid sequence data corresponding to protein-protein complex parts;
obtain a 3D structure model of the protein-protein complex comprising the first protein part and the second protein part predicted by the trained second deep learning model;
determine a low energy score state for the 3D structure models of each of the first protein part, the second protein part, and the protein-protein complex;
generate, based on the low energy score states, an energy score for the 3D structure models of each of the first protein part, the second protein part, and the protein-protein complex; and
determine a score difference between the energy score for the 3D structure model of the protein-protein complex and a sum of the energy scores for the 3D structure models of first protein part and the second protein part, wherein the score difference defines a binding affinity score.

40. A computerized method comprising:
obtaining, from an amino acid sequence database, amino acid sequence data corresponding to a first protein part and a binding site complex, wherein the binding site complex corresponds to a known binding site between the first protein part and a second protein part;
feeding the amino acid sequence data corresponding to the first protein part into a trained deep learning model, wherein the trained deep learning model is trained to predict a 3D structure model based on a first input of amino acid sequence data corresponding to a protein part or a protein-protein complex;
obtaining a 3D structure model of the first protein part predicted by the trained deep learning model;
feeding the amino acid sequence data corresponding to the binding site complex into the trained deep learning model;
obtaining a 3D structure model of the binding site complex predicted by the trained deep learning model;
determining a low energy score state for the 3D structure models of each of the first protein part and the binding site complex;
generating, based on the low energy score states, an energy score for the 3D structure models of each of the first protein part and the binding site complex; and
determining a score difference between the energy score for the 3D structure model of the binding site complex and the energy score for the 3D structure model of first protein part, wherein the score difference defines a binding affinity score.

41. The method of claim 40, wherein the trained deep learning model comprises an AlphaFold multimer model.
